(19) United States
(12) Patent Application Publication
(10) Pub. No.: US 2013/0089854 A1
(43) Pub. Date: Apr. 11, 2013

(54) KIT FOR DOT IMMUNOGOLD DIRECTED FILTRATION ASSAY AND USE THEREOF

(71) Applicant: Yuan Lin, Xi'an (CN)

(72) Inventor: Yuan Lin, Xi'an (CN)

(73) Assignee: XIAN WEITONG BIOSCIENCE LIMITED COMPANY, Xi’an, Shaanxi (CN)

(21) Appl. No.: 13/704,397

(22) PCT Filed: Dec. 10, 2010
(86) PCT No.: PCT/CN2010/002022

§ 371 Date: Dec. 14, 2012

(30) Foreign Application Priority Data
Nov. 25, 2010 (CN) 201010559311.3

(51) Int. Cl. G01N 33/543

(52) U.S. Cl. CPC G01N 33/5436; B82Y 15/00
USPC 435/5; 435/7.23; 435/7.32; 435/7.35; 435/7.37; 435/6.19; 435/7.4; 436/501; 435/7.21; 435/287.2; 422/430; 977/773; 977/920

(57) ABSTRACT

A kit for dot immunogold directed filtration assay including a dot immunogold directed filtration card, a detection probe labeled by nano colloidal gold or latex beads, a negative standard, a positive standard, and a cleaning solution.

### FIELD OF THE INVENTION

The present invention relates to a kit for dot immunogold directed filtration assay, which may be used in fields of scientific research, disease diagnosis and food safety.

### DESCRIPTION OF RELATED ART

Gold immunolabeling assay technique, as a novel immunolabeling technique after occurrence of isotope, fluorescein and enzyme-labeling immunoassay techniques, is convenient and simple, does not require specific devices, and the visibility of the result thereof is excellent. The gold immunolabeling assay technique is suitable for bedside assay, disease investigation and epidemiological surveillance, as well as custom inspection, food safety, breeding industry and the like.

Typical gold immunolabeling assay techniques include bilateral immune-chromatographic assay and dot immunogold filtration assay. In the bilateral immune-chromatographic assay, a sample to be assayed and a colloidal gold labeled probe move laterally on a microporous membrane (for example, a nitrocellulose membrane) by siphonage and meet a capture probe which is pre-coated on the other end of the microporous membrane, and thus gather, thereby presenting a red line visible to naked eyes. In the bilateral immune-chromatographic assay, since the sample to be assayed and the labeled probe move synchronously in a mixture state, the labeled probe may be consumed due to presence of non-specific antibodies in the sample to be assayed during detecting antibodies, thereby deteriorating the detection sensitivity. In this case, a double antigen sandwich method is generally used instead of an indirect antibody method. However, in the double antigen sandwich method, difference between antigenic determinants of a coated antigen and a labeled antigen should be as great as possible so as to avoid a competitive inhibition, thereby requiring higher reagent quality and higher cost, which are main defects of the double antigen sandwich method. In a competitive method, a positive result is obtained when no color appears, which may make it difficult to identify the result in most cases. Therefore, currently the clinical use of the method is limited.

The dot immunogold filtration assay is a method in which a sample to be assayed is allowed to filtrate through a microporous membrane (for example, a nitrocellulose membrane) vertically and is captured by a capture probe coated on the membrane, and a colloidal gold labeled probe is allowed to filtrate through the microporous membrane in the same manner, combine with a captured ligand and gather to form a red dot visible to naked eyes. The method is disadvantageous in that most of the sample to be assayed and the labeled probe are lost from an irrelevant region outside the dot, thereby deteriorating the detection sensitivity. Accordingly, the technique has not been used widely in clinical laboratory test.

### SUMMARY OF THE INVENTION

An object of the present invention is to provide a kit for dot immunogold directed filtration assay having high sensitivity and high accuracy to overcome the disadvantages of the related art.

Another object of the present invention is to provide a use of the kit for dot immunogold directed filtration assay in fields of scientific research, disease diagnosis and food safety.

The kit for dot immunogold directed filtration assay according to the present invention includes a dot immunogold filtration card, a detection probe labeled by nano colloidal gold or latex beads, a negative standard, a positive standard, and a cleaning solution. According to the kit for dot immunogold directed filtration assay, a filtration limiting device is disposed between a microporous membrane and a water absorbent membrane on the basis of the conventional dot immunogold filtration so that a sample to be assayed and a probe labeled by colloidal gold sequentially filtrate along a region covered by a coated probe so as to prevent reagents from being lost from other irrelevant regions and to increase the detection sensitivity.

The object of the present invention is achieved by providing a kit for dot immunogold directed filtration assay including a dot immunogold filtration card, a detection probe labeled by nano colloidal gold or latex beads, a negative standard, a positive standard, and a cleaning solution, wherein the dot immunogold filtration card is a dot immunogold directed filtration card being capable of allowing a sample to be assayed and a probe labeled by colloidal gold to sequentially filtrate along a region covered by a coated probe.

The dot immunogold directed filtration card may be embodied in such two manners that the sample to be assayed and the probe labeled by colloidal gold are allowed to sequentially filtrate along the region covered by the coated probe. In one of the two manners, a filtration limiting layer is disposed between a microporous membrane, having micropores and a function of chromatography, and a water absorbent pad on the basis of the conventional dot immunogold filtration so that the sample to be assayed and the probe labeled by colloidal gold gather towards a central portion of the microporous membrane during passing through the microporous membrane and then filtrate vertically to the water absorbent pad through the region covered by the coated probe in the central portion of the microporous membrane, thereby reducing loss of the reagents in other irrelevant regions and improving the detection sensitivity. Such a dot immunogold directed filtration card consists of four layers of a surface layer, a microporous membrane, a filtration limiting layer, and a water absorbent pad from top to bottom. The surface layer is formed of a water nonabsorbent material and has an opening at a central portion thereof. The microporous membrane has an affinity for protein molecules, and at least one kind of probe is coated on the microporous membrane. The microporous membrane may be, for example, a nitrocellulose membrane, a cellulose acetate membrane, or a PVDF membrane having a pore diameter of 0.2-5 μm. The filtration limiting layer is formed of a water nonabsorbent material, for example, a water nonabsorbent polymer membrane, a double side adhesive or a waterproof coating, and has an opening at a central portion thereof.

In the other of the two manners, the dot immunogold directed filtration card consists of three layers of a surface layer, a microporous membrane, and a water absorbent pad from top to bottom. The surface layer is formed of a water nonabsorbent material and has an opening at a central portion thereof. The microporous membrane has an affinity for protein molecules, and at least one kind of probe is coated on the microporous membrane. The microporous membrane may be, for example, a nitrocellulose membrane, a cellulose acetate membrane, or a PVDF membrane having a pore diameter of 0.2-5 μm. The water absorbent pad has a recess disposed in a central portion of the water absorbent pad and a protrusion disposed in a central portion of the recess and having a size and a shape corresponding to that of the coated probe. A region of the microporous membrane where the probe is coated contacts with the protrusion of the water absorbent pad closely, and other regions of the microporous membrane do not contact with the recess of the water absorbent pad, that is, other regions of the microporous membrane are spaced apart from the recess of the water absorbent pad.

1-2 μL it of capture probe (1 mg/mL) is fixed at the central portion of the microporous membrane by micro spotting or a spraying technique to form a circular spot with a diameter of 2-3 mm or a strip of 2×10 mm², followed by drying at room temperature or 37° C. while blowing air, and the microporous membrane is immersed in 1% bovine serum albumin, calf serum, or skim milk to seal non-binding sites on the membrane, followed by drying at room temperature or 37° C. while blowing air.

The opening in the surface layer of the dot immunogold directed filtration card is circular or oval and has a diameter of 8-12 mm.

The surface layer of the dot immunogold directed filtration card is formed of PVC, PE, PP, PS, ABS plastic, or other water nonabsorbent polymer material.

The water absorbent pad is a water absorbent paperboard, a water absorbent fiberboard, or a water absorbent cotton pad having a thickness of 2-3 mm.

The kit for dot immunogold directed filtration assay may be further provided with a funneled filter membrane fit to the opening of the surface layer of the card and fit to the microporous membrane at the central portion of the surface layer of the card, and may be freely detachable. The filter membrane may be made of micro-scale filter paper with a pore diameter of 0.2-0.45 μm, have an anticoagulant absorbed thereto, and filter formed elements in a sample.

The detection probe labeled by nano colloidal gold or latex beads is a detection probe connected with colloidal gold having a size of 10-40 nm or latex beads, and includes antigen, hapten, antibody, protein molecule, polypeptide, and nucleic acid. These labeled probes bind with a ligand during filtration to gather, presenting a color visible to naked eyes.

The cleaning solution is a phosphate buffer, a Tris-buffer, or a buffer containing Tween 20.

The kit for dot immunogold directed filtration assay according to the present invention may be used for scientific research, disease diagnosis, and food safety, more particularly, for detecting antigen, hapten, antibody, protein molecule, polypeptide, and nucleic acid, detecting drug residue and chemical residue in food, and detecting various markers of diseases including:
infectious diseases including herpes simplex virus 1/2, varicella virus, EB virus, cytomegalo virus, human herpes virus-6/8, influenza virus A/B, parainfluenza virus, respiratory syncytial virus, measles virus, adenovirus, severe acute respiratory syndrome virus, hepatitis, poliovirus, Japanese encephalitis virus, hemorrhagic fever virus, multivalent avian influenza virus, humanimmunodeficiency virus 1/2, rabies virus, meningococcus, Brucella, bacillus tetani, syphilis, mycoplasma pneumoniae, ToxoPlasma gondii, bordetella pertussis, multivalent pneumococcus, multivalent escherichia coli, multivalent corynebacterium diphtheriae, mycobacterium tuberculosis, salmonella typhi, helicobacter pylori, and japonicum;
autoimmune disease including double-stranded DNA, histone, cardiolipin, nuclealar antigen, centromere antigen, myeloperoxidase, protein kinase 3, nucleoprotein, Smith antigen, Jo-1 antigen, Scl-70 antigen, SS-A/B antigen, thyroglobulin, mitochondria antigen, myocardial antigen, smooth muscle antigen, glomerular basement membrane antigen, collagen I-IV, myelin basic protein, proteolipid protein, myelin glia cell glycoprotein, rheumatism factor, and cyclic citrullinated polypeptide;
tumor markers including carcinoma antigen 27.29, carcinoembryonic antigen, carcinoma antigen 19.9, alpha fetal protein, human chorionic gonadotropin, carcinoma antigen 125, anti-cancer antigen 27.29, and prostate cancer specific antigen;
cardiovascular and cerebrovascular diseases including myoglobin, troponin I, high sensitive C-reactive protein, creatine phosphokinase MB, and heart-type fatty acid binding protein;
diseases of poultry and livestock including chicken avian influenza virus, newcastle disease virus, porcine parvovirus, porcine circovirus, porcine foot and mouth disease, porcine blue-ear disease virus, pseudorabies virus, and canine parvovirus;
parasitic diseases including ascariosis of swine, trichinosis, fasciolopsiasis, cysticercosis, sarcocystosis, toxoplasmosis, acanthocephaliasis, pulmonarynematodiasis of swine, trichuridiosis in swine, strongyloidiasis of swine, nodular worm of swine, kidney worm disease of swine, hyostrongylus rubidus disease of swine, piroplasmosis of cattle, bovine coccidiosis, sarcocystosis of cattle, toxoplasmosis of cattle, trichomonas foetus of cattle, cysticercosis bovis of cattle, echinococcosis of cattle, fascioliasis hepatica of cattle, cattle schistosomiasis, fascioliasis hepatica of sheep, sheep schistosomiasis, paramphistomiasis of sheep, dicrocoeliasis of sheep, coenurosis cerebralis of sheep, echinococcosis of sheep, tenuicollis cysticercosis of sheep, gastrointestinal nematodiasis of sheep, pulmonarynematodiasis of sheep, taeniasis of sheep, haemosporidiasis of sheep, coccidiosis of sheep, and theileriidae of sheep;
detection of potency of vaccine including: smallpox vaccine, poliomyelitis vaccine, measles vaccine, adenovirus vaccine, yellow fever vaccine, rubella vaccine, mumps vaccine, hepatitis A vaccine, influenza vaccine, varicella vaccine, rotavirus vaccine, Japanese encephalitis vaccine, rabies vaccine, epidemic hemorrhagic fever vaccine, bacillus calmette guerin vaccine, typhoid vaccine, typhus vaccine, cholera vaccine, brucellosis vaccine, anthrax vaccine, shigella vaccine, pertussis vaccine, plague vaccine, streptococcus pneumoniae vaccine, salmonella Vi polysaccharide vaccine, meningococcus A/C vaccine, shigella vaccine, pertussis vaccine, plague vaccine, streptococcus pneumoniae vaccine, salmonella Vi polysaccharide vaccine, meningococcus A/C vaccine, haemophilus influenzae vaccine, diphtheria vaccine, and SARS vaccine.

Advantages and positive effects of the present invention:

(1) The dot immunogold directed filtration assay according to the present invention significantly improves the detection sensitivity compared to the conventional dot immunogold filtration.

(2) The kit for dot immunogold directed filtration assay according to the present invention may be easily fabricated, consumes fewer reagents, and has a broader detection limit and better operability compared to the immune-chromatographic assay.

(3) The kit for dot immunogold directed filtration assay and the use thereof according to the present invention have a low cost and are suitable for being implemented in a large scale.

### BRIEF DESCRIPTION OF THE DRAWINGS

FIG. 1 is a structural schematic view showing a dot immunogold directed filtration card having four layers according to the present invention;

FIG. 2 is a schematic view showing a filtration direction of the dot immunogold directed filtration card having four layers according to the present invention;

FIG. 3 is a structural schematic view showing a dot immunogold directed filtration card having three layers according to the present invention;

FIG. 4 is a schematic view showing a filtration direction of the dot immunogold directed filtration card having three layers according to the present invention; and

FIG. 5 is a schematic view showing a positive result assayed by a dot immunogold directed filtration card according to the present invention.

### DETAILED DESCRIPTION OF THE PREFERRED EMBODIMENTS

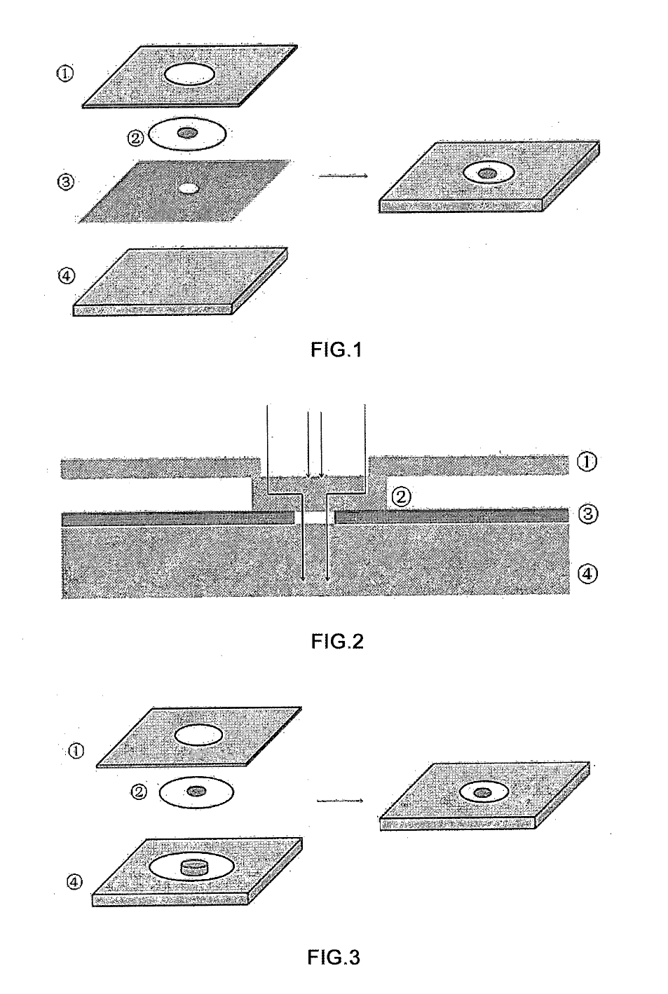

As shown in FIG. 1, a dot immunogold directed filtration card according to the present invention consists of four layers of a surface layer \textcircled{1} formed of a water nonabsorbent material, a microporous membrane \textcircled{2} coated with a capture probe, a filtration limiting membrane \textcircled{3} formed of a water nonabsorbent material, and a water absorbent pad \textcircled{4} from top to bottom, and the four layers are combined to form the dot immunogold directed filtration card according to the present invention.

As shown in FIG. 2, the dot immunogold directed filtration card is placed horizontally at room temperature. A blood serum or plasma to be assayed is diluted properly (1:(5-10)), and 50-100 μL of such a diluted sample of blood serum or plasma is dropped onto the microporous membrane at a central portion of the card by using a micropipettor. After filtration of the diluted sample is completed, 50-100 μL of PBS buffer is dropped. After filtration of the PBS buffer is completed, 50-100 μL of detection probe labeled by gold is dropped. After filtration of the detection probe is completed, 50-100 μL of PBS buffer is dropped. Positive and negative standards are assayed by the same method, obtaining results as a quality control in assay. Color reactions in the region of the capture probe are observed by naked eyes. A red spot is identified as a positive result and absence of color reaction is identified as a negative result. As for result determination, if each of the results of the positive control and the assayed sample is positive, the assayed sample is identified to be positive; if the result of the positive control is positive and the result of the assayed sample is negative, the assayed sample is identified to be negative; and if the result of the positive control is negative, there is an error in the test and the test should be repeated. The whole test takes about 3-5 minutes. The sample to be assayed may be pre-diluted in a gradient, and a titer of the sample may be analyzed quantitatively.

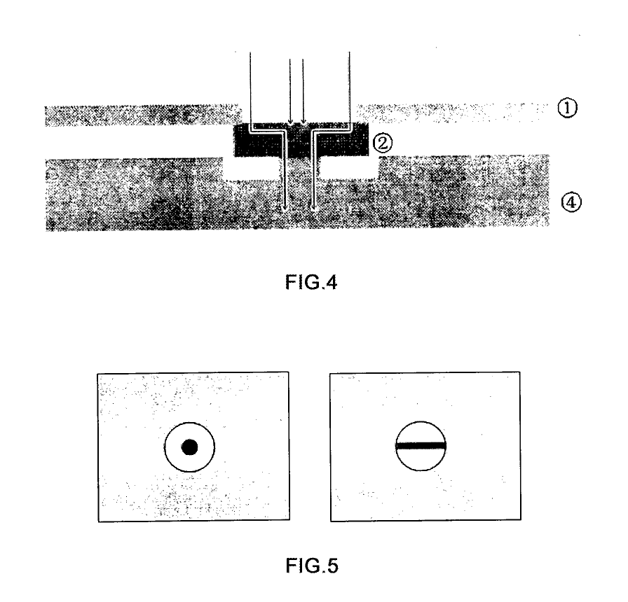

As shown in FIGS. 3 and 4, a dot immunogold directed filtration card according to the present invention consists of three layers of a surface layer \textcircled{1} formed of a water nonabsorbent material, a microporous membrane \textcircled{2} coated with a capture probe, and a water absorbent pad \textcircled{4} from top to bottom, and the three layers are combined to form the dot immunogold directed filtration card according to the present invention. The water absorbent pad \textcircled{4} has a recess disposed in a central portion of the water absorbent pad \textcircled{4} and a protrusion disposed in a central portion of the recess and having a size and a shape corresponding to that of the coated probe. A region of the microporous membrane where the probe is coated contacts with the protrusion of the water absorbent pad closely, and other regions of the microporous membrane are spaced apart from (do not contact with) the recess of the water absorbent pad to ensure directed filtration of a solution in a direction indicated by an arrow and enrich the sample, thereby improving the detection sensitivity.

A filter membrane may be further disposed on the microporous membrane at a central portion of the dot immunogold directed filtration card according to the present invention. 5-10 μL of blood serum, blood plasma or whole blood sample is dropped onto the filter membrane by using a micropipettor, followed by dropping 50-100 μL of PBS buffer. After filtration of the PBS buffer is completed, 50-100 μL of detection probe labeled by gold is dropped, and subsequent steps are the same as above.

FIG. 5 is a schematic view showing a positive result assayed by a dot immunogold directed filtration card according to the present invention. As shown in FIG. 5, a circular spot \textcircled{1} or a strip \textcircled{2} is represented according to the shape of the region coated by the capture probe.

### Example 1

### Fabrication of a Kit for Dot Immunogold Directed Filtration Assay

The surface layer of the dot immunogold directed filtration card was a white PVC card having a size of 5×4×0.5 cm³ and including a circular hole with a diameter of 0.8 cm at a center of the surface layer. The microporous membrane of the filtration card was a NC membrane having a diameter of 1.2 cm and a pore size of 0.45 μm. 1-2 μL of capture probe (1 mg/mL) was fixed at a central portion of the microporous membrane by micro spotting or a spraying technique to form a circular spot with a diameter of 2 mm or a strip, followed by drying at room temperature or 37° C. while blowing air, and the microporous membrane was immersed in 1% bovine serum albumin for 1 minute, followed by drying at room temperature or 37° C. for 20 minutes while blowing air. The filtration limiting layer of the filtration card was a double side adhesive thin film having a size of 5×4×0.5 cm³ and including a circular hole with a diameter of 2 mm or a strip, which was positioned at a center of the double side adhesive thin film to correspond to a region where the capture probe was coated at the central portion of the microporous membrane. The water absorbent pad of the filtration card was a water absorbent paperboard having a size of 5×4×0.3 cm³. The above four layers were sequentially adhered closely by adhesives, and the formed card was stored hermetically in an aluminum foil pouch.

The positive and negative controls were respectively positive and negative samples calibrated by using standards. The capture probe labeled by gold was a detection probe labeled by nano colloidal gold having a diameter of 20 nm. The cleaning solution was a PBS buffer having a concentration of 0.1 M.

Alternatively, the above double side adhesive thin film as the filtration limiting layer was omitted, and the water absorbent pad of the filtration card was punched at a center thereof to have a circular recess having a diameter of 10 mm and a depth of 1.5 mm and a cylindrical protrusion having a diameter of 2 mm at a center of the recess. The water absorbent pad, the above microporous membrane and the PVC card were sequentially adhered closely from bottom to top, and the formed card was stored hermetically in an aluminum foil pouch.

### Example 2

### Fabrication of a Two-Dot Immunogold Directed Filtration Card

The two-dot immunogold directed filtration card was used to simultaneously show a positive control and a detection spot in a single detection hole. The surface layer of the dot immunogold directed filtration card was a white PVC card having a size of 5×4×0.5 cm³ and including a circular or oval hole with a diameter of 1 cm at a center of the surface layer. The microporous membrane of the filtration card was a NC membrane having a diameter of 1.5 cm and a pore size of 0.45 μm. 1-2 μL of anti-probe labeled by gold antibody (1 mg/mL) and 1-2 μL of capture probe (1 mg/mL) were respectively fixed at a central portion of the microporous membrane by micro spotting or a spraying technique with an interval of 4 mm therebetween, thereby forming circular spots each having a diameter of 2 mm or strips, followed by drying at room temperature or 37° C. while blowing air, and the microporous membrane was immersed in 1% bovine serum albumin for 1 minute, followed by drying at room temperature or 37° C. for 20 minutes while blowing air. The filtration limiting layer of the filtration card was a double side adhesive thin film having a size of 5×4×0.5 cm³ and including two circular holes each having a diameter of 2 mm or two strips, which were positioned at a center of the double side adhesive thin film to correspond to two regions where the capture probes were coated at the central portion of the microporous membrane. The water absorbent pad of the filtration card was a water absorbent paperboard having a size of 5×4×0.3 cm³. The above four layers were sequentially adhered closely by adhesives to complete the fabrication of the card, and the formed card was stored hermetically in an aluminum foil pouch.

### Example 3

### Fabrication of a Dot Immunogold Directed Filtration Card for Detecting an Antigen by a Double Antibody Method

The surface layer of the dot immunogold directed filtration card was a white PVC card having a size of 5×4×0.5 cm³ and including a circular hole with a diameter of 0.8 cm at a center of the surface layer. The microporous membrane of the filtration card was a NC membrane having a diameter of 1.2 cm and a pore size of 0.45 μm. 1-2 μL of capture antibody (1 mg/mL) was fixed at a central portion of the microporous membrane by micro spotting or a spraying technique to form a circular spot with a diameter of 2 mm or a line or a ring, followed by drying at room temperature or 37° C. while blowing air, and the microporous membrane was immersed in 1% bovine serum albumin for 1 minute, followed by drying at room temperature or 37° C. for 20 minutes while blowing air. Other operations were the same as in Example 1.

### Example 4

### Fabrication of a Dot Immunogold Directed Filtration Card for Detecting an Antibody by an Indirect Antibody Method

The surface layer of the dot immunogold directed filtration card was a white PVC card having a size of 5×4×0.5 cm³ and including a circular hole with a diameter of 0.8 cm at a center of the surface layer. The microporous membrane of the filtration card was a NC membrane having a diameter of 1.2 cm and a pore size of 0.45 μm. 1-2 μL of antigen (1 mg/mL) as a capture probe was fixed at a central portion of the microporous membrane by micro spotting or a spraying technique to form a circular spot with a diameter of 2 mm or a line or a ring, followed by drying at room temperature or 37° C. while blowing air, and the microporous membrane was immersed in 1% bovine serum albumin for 1 minute, followed by drying at room temperature or 37° C. for 20 minutes while blowing air. Other operations were the same as in Example 1.

### Example 5

### Detection of Hepatitis B Virus Antigen by a Double Antibody Method

The kit included dot immunogold directed filtration cards respectively coated by antibodies specific for hepatitis B surface antigen (HbsAg), hepatitis B core antigen (HbcAg) and hepatitis B e antigen (HbeAg), positive and negative control serums of hepatitis B virus, antibodies specific for hepatitis B surface antigen, hepatitis B core antigens and hepatitis B e antigens labeled by colloidal gold having a size of 20 nm, and a PBS cleaning solution. A blood serum or plasma to be assayed was diluted with the PBS buffer in a ratio of 1:10, and 100 μL of such a diluted sample was dropped onto the microporous membrane at a central portion of the card by using a micropipettor. After filtration of the diluted sample was completed, 100 μL of PBS buffer was dropped for cleaning After filtration of the PBS buffer was completed, 50 μL of probe labeled by gold was dropped. After filtration of the probe labeled by gold was completed, 100 μL of PBS buffer was dropped for cleaning Then, the color of the spot was observed. The positive and negative control serums were assayed in parallel to obtain results as quality controls. Furthermore, results obtained from the dot immunogold directed filtration card according to Example 5 was compared with that of a conventional dot immunogold filtration card. Detection results for 20 hepatitis B positive serums and 20 normal controls show that the dot inmmunogold directed filtration assay according to the present invention and the conventional dot immunogold filtration assay had sensitivities of 95% and 40%, respectively, and specificities of 100%.

### Example 6

### Detection of Hepatitis C Virus Antibody by an Indirect Antibody Method

The kit included dot immunogold directed filtration cards coated by recombinant hepatitis C virus nonstructural antigen (HCVns) and core antigen (HCVc), positive and negative control serums of hepatitis C virus, a goat anti-human IgG antibody labeled by colloidal gold having a size of 20 nm, and a PBS cleaning solution. Operations in Example 6 were the same as in Example 5. Detection results for 20 hepatitis C serums and 20 normal controls show that the dot inmmunogold directed filtration assay according to the present invention and the conventional dot immunogold filtration assay had sensitivities of 85% and 45%, respectively, and specificities of 100%.

### Example 7

### Detection of Humanimmunodeficiency Virus (HIV) Antibody by an Indirect Antibody Method

The kit included a dot immunogold directed filtration card coated by recombinant HIV1+2 antigen, HIV positive and negative control serums, a goat anti-human IgG antibody labeled by colloidal gold having a size of 20 nm, and a PBS cleaning solution. Operations in Example 7 were the same as in Example 5. Detection results for 20 HIV serums and 20 normal controls show that the dot inmmunogold directed filtration assay according to the present invention and the conventional dot immunogold filtration assay had sensitivities of 95% and 40%, respectively, and specificities of 100%.

### Example 8

### Detection of Treponema Pallidum Antibody by an Indirect Antibody Method

The kit included a dot immunogold directed filtration card coated by recombinant treponema pallidum antigen, syphilis positive and negative control serums, a goat anti-human IgG antibody labeled by colloidal gold having a size of 20 nm, and a PBS cleaning solution. Operations in Example 8 were the same as in Example 5. Detection results for 20 syphilis serums and 20 normal controls show that the dot inmmunogold directed filtration assay according to the present invention and the conventional dot immunogold filtration assay had sensitivities of 95% and 20%, respectively, and specificities of 100%.

### Example 9

### Detection of Epidemic Hemorrhagic Fever Virus (Hantan virus) Antibody

The kit included a dot immunogold directed filtration card coated by recombinant epidemic hemorrhagic fever virus, positive and negative control serums of epidemic hemorrhagic fever virus, goat anti-human IgG+IgM antibodies labeled by colloidal gold having a size of 20 nm, and a PBS cleaning solution. Operations in Example 9 were the same as in Example 5. Detection results for 20 epidemic hemorrhagic fever virus serums and 20 normal controls show that the dot inmmunogold directed filtration assay according to the present invention and the conventional dot immunogold filtration assay had sensitivities of 90% and 15%, respectively, and specificities of 100%.

### Example 10

### Detection of Autoimmune Antibodies

The kit included dot immunogold directed filtration cards respectively coated by double-stranded DNA, histone, cardiolipin, nuclealar antigen, centromere antigen, myeloperoxidase, protein kinase 3, nucleoprotein, Smith antigen, Jo-1 antigen, Scl-70 antigen, SS-A/B antigen, thyroglobulin, mitochondria antigen, myocardial antigen, smooth muscle antigen, glomerular basement membrane antigen, collagen I-IV, myelin basic protein, proteolipid protein, myelin glia cell glycoprotein, rheumatism factor and cyclic citrullinated polypeptide (CCP), control serums relevant to the autoimmune diseases and normal control serums, a goat anti-human IgG antibody labeled by colloidal gold having a size of 20 nm, and a PBS cleaning solution. Operations in Example 10 were the same as in Example 5. Detection results for anti-CCP antibody of 20 rheumatoid arthritis serums and 20 normal controls show that the dot inmmunogold directed filtration assay according to the present invention and the conventional dot immunogold filtration assay had sensitivities of 95% and 30%, respectively, and specificities of 100%.

### Example 11

### Detection of Tumor Markers by using a Dot Immunogold Directed Filtration Card

The kit included dot immunogold directed filtration cards respectively coated by antibodies specific for carcinoma antigen 27.29 (CA27.29), carcinoembryonic antigen (CEA), carcinoma antigen 19.9 (CA19.9), alpha fetal protein (AFP), human chorionic gonadotropin (b-hCG), carcinoma antigen 125 (CA125) and prostate cancer specific antigen (PSA), positive and negative control serums relevant to the tumors, antibodies relevant to the tumor markers labeled by colloidal gold having a size of 20 nm, and a PBS cleaning solution. Operations in Example 11 were the same as in Example 5. Detection results for 20 PSA serums and 20 normal controls show that the dot inmmunogold directed filtration assay according to the present invention and the conventional dot immunogold filtration assay had sensitivities of 65% and 10%, respectively, and specificities of 100%.

### Example 12

### Detection of Cardiovascular and Cerebrovascular Disease Biomarkers by using a Dot Immunogold Directed Filtration Card

The kit included dot immunogold directed filtration cards respectively coated by antibodies specific for myoglobin, troponin I, high sensitive C-reactive protein, creatine phosphokinase MB and heart-type fatty acid binding protein, positive and negative control serums, antibodies relevant to the cardiovascular and cerebrovascular disease biomarkers labeled by colloidal gold having a size of 20 nm, and a PBS cleaning solution. Operations in Example 12 were the same as in Example 5. Detection results for high sensitive C-reactive protein of 20 patient serums and 20 normal controls show that the dot inmmunogold directed filtration assay according to the present invention and the conventional dot immunogold filtration assay had sensitivities of 70% and 15%, respectively, and specificities of 100%.

### Example 13

### Detection of Melamine Residue in Food

The kit included a dot immunogold directed filtration card coated by a rabbit anti melamine antibody, positive and negative controls, a goat anti melamine antibody labeled by colloidal gold having a size of 20 nm, and a PBS cleaning solution. Operations in Example 13 were the same as in Example 5. Detection results for 20 positive samples and 20 normal controls show that the dot inmmunogold directed filtration assay according to the present invention and the conventional dot immunogold filtration assay had sensitivities of 100% and 35%, respectively, and specificities of 100%.

### Example 14

### Detection of Poultry Infectious Diseases

The kit included dot immunogold directed filtration cards coated by inactivated chicken avian influenza virus and newcastle disease virus antigens, chicken avian influenza serum, newcastle disease serum and normal chicken serum controls, goat anti-chicken IgM and IgG antibodies labeled by colloidal gold having a size of 20 nm, and a PBS cleaning solution. Operations in Example 14 were the same as in Example 5. Detection results for 20 chicken avian influenza serums and 20 normal serum controls show that the dot inmmunogold directed filtration assay according to the present invention and the conventional dot immunogold filtration assay had sensitivities of 90% and 25%, respectively, and specificities of 100%.

### Example 15

### Detection of Livestock Infectious Diseases

The kit included dot immunogold directed filtration cards coated by inactivated porcine parvovirus, porcine circovirus, porcine foot and mouth disease and porcine blue-ear disease virus antigens, infected serum and normal serum controls, goat anti-swine IgM and IgG antibodies labeled by colloidal gold having a size of 20 nm, and a PBS cleaning solution. Operations in Example 15 were the same as in Example 5. Detection results for 20 serums infected with porcine parvovirus and 20 normal serum controls show that the dot inmmunogold directed filtration assay according to the present invention and the conventional dot immunogold filtration assay had sensitivities of 90% and 20%, respectively, and specificities of 100%.

1-14. (canceled)

15. A kit for dot immunogold directed filtration assay, comprising a dot immunogold filtration card, a detection probe labeled by nano colloidal gold or latex beads, a negative standard, a positive standard, and a cleaning solution, wherein the dot immunogold filtration card is a dot immunogold directed filtration card being capable of allowing a sample to be assayed and a probe labeled by colloidal gold to sequentially filtrate along a region covered by a coated probe.

16. The kit for dot immunogold directed filtration assay of claim 15, wherein the dot immunogold directed filtration card consists of four layers of a surface layer, a microporous membrane, a filtration limiting layer, and a water absorbent pad from top to bottom, the surface layer is formed of a water nonabsorbent material and has an opening at a central portion thereof, the microporous membrane has an affinity for protein molecules, at least one kind of probe is coated on the microporous membrane, the filtration limiting layer is formed of a water nonabsorbent material and has an opening at a central portion thereof, and the opening of the filtration limiting layer corresponds to the coated probe and has a shape fit to that of the coated probe.

17. The kit for dot immunogold directed filtration assay of claim 16, wherein the filtration limiting layer of the dot immunogold directed filtration card is formed of PVC, PE, PP, PS, ABS plastic, a double side adhesive, or a waterproof coating.

18. The kit for dot immunogold directed filtration assay of claim 15, wherein the dot immunogold directed filtration card consists of three layers of a surface layer, a microporous membrane, and a water absorbent pad from top to bottom, the surface layer is formed of a water nonabsorbent material and has an opening at a central portion thereof, the microporous membrane has an affinity for protein molecules, at least one kind of probe is coated on the microporous membrane, the water absorbent pad has a recess disposed in a central portion thereof and a protrusion disposed in a central portion of the recess and having a size and a shape corresponding to that of the coated probe, the region covered by the coated probe closely contacts with the protrusion of the water absorbent pad, and other regions of the microporous membrane do not contact with the water absorbent pad.

19. The kit for dot immunogold directed filtration assay of claim 16, wherein a capture probe is fixed at a central portion of the microporous membrane by micro spraying to form a circular spot with a diameter of 2-3 mm or a strip of 2×10 mm².

20. The kit for dot immunogold directed filtration assay of claim 18, wherein a capture probe is fixed at a central portion of the microporous membrane by micro spraying to form a circular spot with a diameter of 2-3 mm or a strip of 2×10 mm².

21. The kit for dot immunogold directed filtration assay of claim 16, wherein the opening of the surface layer of the dot immunogold directed filtration card is circular or oval and has a diameter of 8-12 mm.

22. The kit for dot immunogold directed filtration assay of claim 18, wherein the opening of the surface layer of the dot immunogold directed filtration card is circular or oval and has a diameter of 8-12 mm.

23. The kit for dot immunogold directed filtration assay of claim 21, wherein the surface layer of the dot immunogold directed filtration card is formed of PVC, PE, PP, PS, or ABS plastic.

24. The kit for dot immunogold directed filtration assay of claim 22, wherein the surface layer of the dot immunogold directed filtration card is formed of PVC, PE, PP, PS, or ABS plastic.

25. The kit for dot immunogold directed filtration assay of claim 16, wherein the microporous membrane of the dot immunogold directed filtration card is a nitrocellulose membrane, a cellulose acetate membrane, or a PVDF membrane and has a pore diameter of 0.2-5 μm.

26. The kit for dot immunogold directed filtration assay of claim 18, wherein the microporous membrane of the dot immunogold directed filtration card is a nitrocellulose membrane, a cellulose acetate membrane, or a PVDF membrane and has a pore diameter of 0.2-5 μm.

27. The kit for dot immunogold directed filtration assay of claim 16, wherein the water absorbent pad is a water absorbent paperboard, a water absorbent fiberboard, a water absorbent cotton pad, a water absorbent resin, or a water absorbent composite material having a thickness of 2-3 mm.

28. The kit for dot immunogold directed filtration assay of claim 18, wherein the water absorbent pad is a water absorbent paperboard, a water absorbent fiberboard, a water absorbent cotton pad, a water absorbent resin, or a water absorbent composite material having a thickness of 2-3 mm.

29. The kit for dot immunogold directed filtration assay of claim 16, further comprising a funneled filter membrane being fit to the opening of the surface layer of the card, made of micro-scale filter paper with a pore diameter of 0.2-0.45 μm, having an anticoagulant absorbed thereto, and filtering formed elements in a sample.

30. The kit for dot immunogold directed filtration assay of claim 18, further comprising a funneled filter membrane being fit to the opening of the surface layer of the card, made of micro-scale filter paper with a pore diameter of 0.2-0.45 μm, having an anticoagulant absorbed thereto, and filtering formed elements in a sample.

31. The kit for dot immunogold directed filtration assay of claim 15, wherein the detection probe labeled by nano colloidal gold or latex beads is a detection probe connected with colloidal gold having a size of 10-40 nm or latex beads, and comprises antigen, hapten, antibody, protein molecule, polypeptide, and nucleic acid, and the labeled probes bind with a ligand during filtration to gather, presenting a color visible to naked eyes.

32. The kit for dot immunogold directed filtration assay of claim 15, wherein the cleaning solution is a phosphate buffer, a Tris-buffer, or a buffer containing Tween 20.

33. A use of the kit for dot immunogold directed filtration assay of claim 15, in fields of scientific research, food safety, and disease diagnosis.

34. The use of claim 33, comprising detecting antigen, hapten, antibody, protein molecule, polypeptide, and nucleic acid, detecting drug residue and chemical residue in food, or detecting various markers of diseases comprising:
infectious diseases comprising herpes simplex virus 1/2, varicella virus, EB virus, cytomegalo virus, human herpes virus-6/8, influenza virus A/B, parainfluenza virus, respiratory syncytial virus, measles virus, adenovirus, severe acute respiratory syndrome virus, hepatitis, poliovirus, Japanese encephalitis virus, hemorrhagic fever virus, multivalent avian influenza virus, humanimmunodeficiency virus 1/2, rabies virus, meningococcus, Brucella, bacillus tetani, syphilis, mycoplasma pneumoniae, ToxoPlasma gondii, bordetella pertussis, multivalent pneumococcus, multivalent escherichia coli, multivalent corynebacterium diphtheriae, mycobacterium tuberculosis, salmonella typhi, helicobacter pylori, and japonicum;
autoimmune disease comprising double-stranded DNA, histone, cardiolipin, nuclealar antigen, centromere antigen, myeloperoxidase, protein kinase 3, nucleoprotein, Smith antigen, Jo-1 antigen, Scl-70 antigen, SS-A/B antigen, thyroglobulin, mitochondria antigen, myocardial antigen, smooth muscle antigen, glomerular basement membrane antigen, collagen I-IV, myelin basic protein, proteolipid protein, myelin glia cell glycoprotein, rheumatism factor, and cyclic citrullinated polypeptide;
tumor markers comprising carcinoma antigen 27.29, carcinoembryonic antigen, carcinoma antigen 19.9, alpha fetal protein, human chorionic gonadotropin, carcinoma antigen 125, anti-cancer antigen 27.29, and prostate cancer specific antigen;
cardiovascular and cerebrovascular diseases comprising myoglobin, troponin I, high sensitive C-reactive protein, creatine phosphokinase MB, and heart-type fatty acid binding protein;
diseases of poultry and livestock comprising chicken avian influenza virus, newcastle disease virus, porcine parvovirus, porcine circovirus, porcine foot and mouth disease, porcine blue-ear disease virus, pseudorabies virus, and canine parvovirus;
parasitic diseases comprisingascariosis of swine, trichinosis, fasciolopsiasis, cysticercosis, sarcocystosis, toxoplasmosis, acanthocephaliasis, pulmonarynematodiasis of swine, trichuridiosis in swine, strongyloidiasis of swine, nodular worm of swine, kidney worm disease of swine, hyostrongylus rubidus disease of swine, piroplasmosis of cattle, bovine coccidiosis, sarcocystosis of cattle, toxoplasmosis of cattle, trichomonas foetus of cattle, cysticercosis bovis of cattle, echinococcosis of cattle, fascioliasis hepatica of cattle, cattle schistosomiasis, fascioliasis hepatica of sheep, sheep schistosomiasis, paramphistomiasis of sheep, dicrocoeliasis of sheep, coenurosis cerebralis of sheep, echinococcosis of sheep, tenuicollis cysticercosis of sheep, gastrointestinal nematodiasis of sheep, pulmonarynematodiasis of sheep, taeniasis of sheep, haemosporidiasis of sheep, coccidiosis of sheep, and theileriidae of sheep;
detection of potency of vaccine comprising: smallpox vaccine, poliomyelitis vaccine, measles vaccine, adenovirus vaccine, yellow fever vaccine, rubella vaccine, mumps vaccine, hepatitis A vaccine, influenza vaccine, varicella vaccine, rotavirus vaccine, Japanese encephalitis vaccine, rabies vaccine, epidemic hemorrhagic fever vaccine, bacillus calmette guerin vaccine, typhoid vaccine, typhus vaccine, cholera vaccine, brucellosis vaccine, anthrax vaccine, shigella vaccine, pertussis vaccine, plague vaccine, streptococcus pneumoniae vaccine, salmonella Vi polysaccharide vaccine, meningococcus A/C vaccine, shigella vaccine, pertussis vaccine, plague vaccine, streptococcus pneumoniae vaccine, salmonella Vi polysaccharide vaccine, meningococcus A/C vaccine, haemophilus influenzae vaccine, diphtheria vaccine, and SARS vaccine.
